(19) United States
(12) Patent Application Publication
(10) Pub. No.: US 2003/0163100 A1
(43) Pub. Date: Aug. 28, 2003

(54) Nasal jejunum feeding tube

(76) Inventors: Rebecca DeLegge, Mt. Pleasant, SC (US); Mark DeLegge, Mt. Pleasant, SC (US)

Correspondence Address: B. Craig Killough, Barnwell Whaley Patterson & Helms, LLC, P.O. Drawer H, Charleston, SC, 29402, US

(21) Appl. No.: 10/340,241

(22) Filed: Jan. 10, 2003

Related U.S. Application Data
(60) Provisional application No. 60/347,367, filed on Jan. 10, 2002.

(51) Int. Cl. A61M 31/00

(52) U.S. Cl. 604/275

(57) ABSTRACT

A device for assisting the guiding of an Nasal Gastric Jejunum (NGJ) or Nasal Jejunum (NJ) tube through the stomach and into the jejunum. The device has a tip that is present on a forward end of a tube. The tip is formed to allow the forward end of the tube to be guided by forceps while also allowing a stiffening stylet, which is inserted into the tube to be used to guide the tube. The tip prevents the stylet from extending beyond the forward end of the tube.

### FIELD OF THE INVENTION

This invention relates to feeding tubes generally, and is more specifically directed to a device for directing a feeding tube which is inserted through the nose or mouth for supplying nutrition jejunally or gastricly.

### BACKGROUND OF THE INVENTION

Nasal Gastric Jejunum (NGJ) and Nasal Jejunum (NJ) tubes are known and used in the art. NGJ and NJ tubes are used to provide nutrition to patients who are unable to swallow, or are otherwise unable to receive food by normal bodily processes due to disease or injury. NGJ tubes or NJ tube are positioned within the stomach and extend into the jejunum. NGJ tubes are typically used for short term feeding of patients. Feeding directly into the jejunum prevents aspiration of food, and problems associated therewith, such as pneumonia.

The tube may also have one or more apertures that allow feeding gastricly, in which case it is an NGJ tube. The tube typically has an outer tube for gastric feeding, with an eccentric smaller tube positioned within the larger tube that extends to the jejunum.

In the prior art, NGJ and NJ tubes are inserted through the nose, and travel through the esophagus, the stomach, the pylorus, and the duodenum, with an end having an aperture resting in the jejunum. The difficulty in the positioning process is guiding the tube from the stomach into the pylorus, since the stomach is a relatively large cavity, while the pylorus presents a restriction as the leading end of the tube exits the stomach. Further, the presence of an angle requires the tube to make a turn so that the leading end can enter the pylorus pass to the duodenum. Insertion of the tube along with an endoscope allows the viewing of the position of the tube, but the scope does not guide the tube, particularly as it approaches the pylorus.

### SUMMARY OF THE PRESENT INVENTION

The present invention is a device for assisting the guiding of an NGJ or NJ tube through the stomach and into the jejunum. The tube has a tip that is present on a forward end of the tube. The tip is formed to allow the forward end of the tube to be guided or pulled by a forceps or other pushing device outside and parallel to the axis of the NG/NGJ, while also allowing a stiffening stylet, which is inserted into the tube, and is used to guide the tube. The tip prevents the stylet from extending beyond the forward end of the tube.

### BRIEF DESCRIPTION OF THE DRAWINGS

The drawing figures depict the device, and the steps of inserting a NGJ or NJ tube, while using the device according to the method of the present invention.

### DETAILED DESCRIPTION OF THE PREFERRED EMBODIMENT

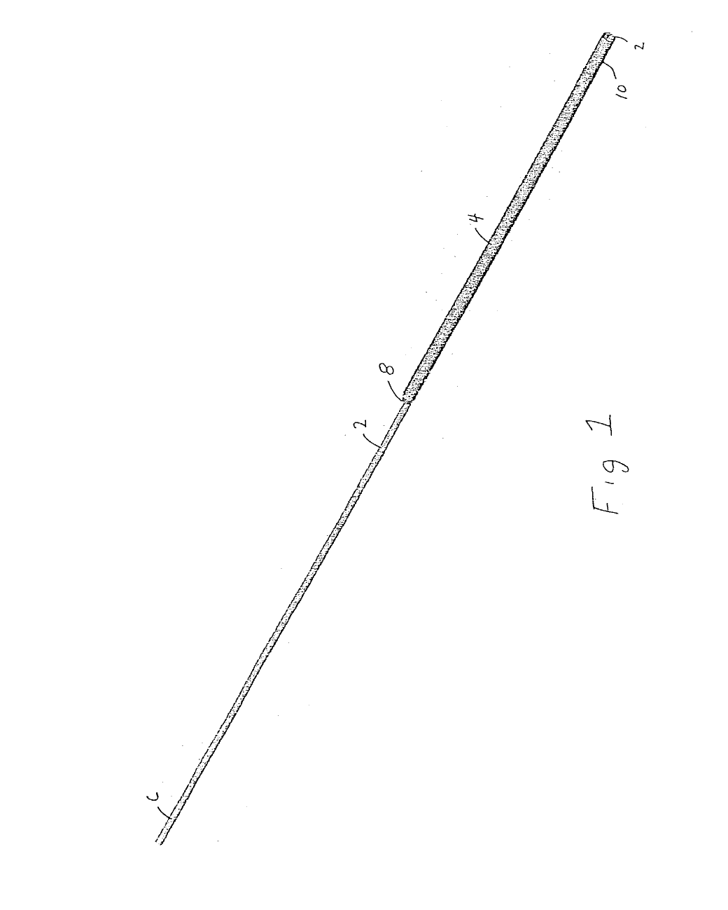

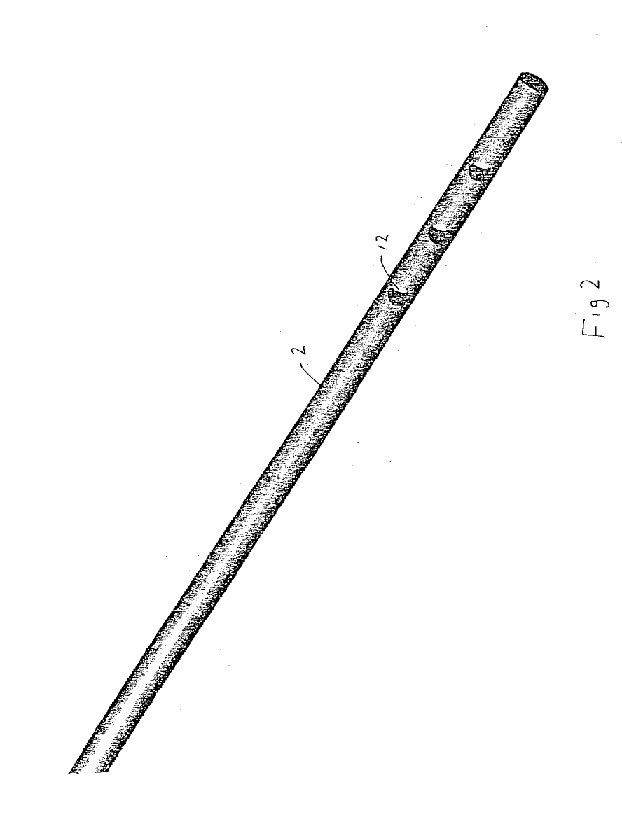

An NGJ or NJ tube is shown in FIG. 1. The tube as shown is made up of a longer tube 2 of smaller diameter, and a shorter tube. The longer tube is present within the shorter tube 4. The shorter tube has a larger diameter, and has a sufficiently large void therein for receiving the larger tube within the void. The forward end 6 of the longer tube is positioned within the jejunum. End 8 of the shorter tube is positioned within the stomach, and opens into the stomach for gastric feeding. The longer tube is eccentrically positioned within the shorter tube at the opposite end 10.

The longer tube 2 may have at least one orifice 12 that permits the liquid nutritional product to exit the tube.

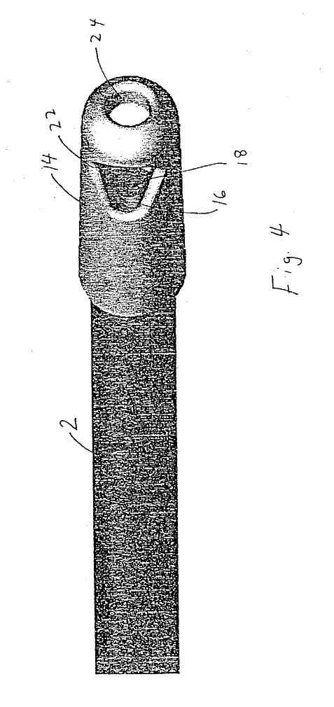

The present invention provides a tip 14 for a forward end of a tube. FIG. 4. The tip fits over the tube with a frictional fit that holds the tip securely in place on the tube. The tip could be attached to the tube with appropriate inert adhesives, or with fasteners. Alternatively, a tube could be formed so as to provide the tip structure.

Formed within the tip, and along a length of the tip, is a divider 16. The divider has an orifice 18 herein. The orifice is smaller than the outside diameter of a stiffening stylet 20 that is inserted through the longer tube 2 for directing and positioning the tube. This divider is a restriction at or near the end of the forward opening of tube 2. The divider prevents the stiffening stylet from extending beyond the end of the tube, and provides resistance to the stiffening stylet, so that the stiffening stylet can direct and position the forward end of the tube. The divider has an orifice therein which allows liquid nutritional products to exit the tube at the forward end of the tube that is within the jejunum.

In the preferred embodiment, the tip has a notch 22 on each side thereof. In the embodiment as shown, the notch comprises an orifice that extends from the outside surface of the tip to the inside of the tip, so that liquid nutritional products transported through the tube can exit through the notch. The void in the notch communicates with the tube to allow the nutritional product to be transported through the orifice 18 and the orifice in the notch.

Two notches are preferred, with the notches positioned on opposite sides of the tip. The use of opposed notches allows the tip to receive forceps, or similar grasping devices having jaws, for direction of the tip as it is positioned. In particular, forceps or similar devices may be used to direct the tube from the stomach into, and through, the pylorus. Triangular or bell shaped notches as shown in the drawing figures are particularly well suited for receiving forceps or similar tools.

The tip has an orifice 24 on the forward end thereof. This orifice also allows the liquid nutritional product to exit the tube and the tip, and to enter the jejunum.

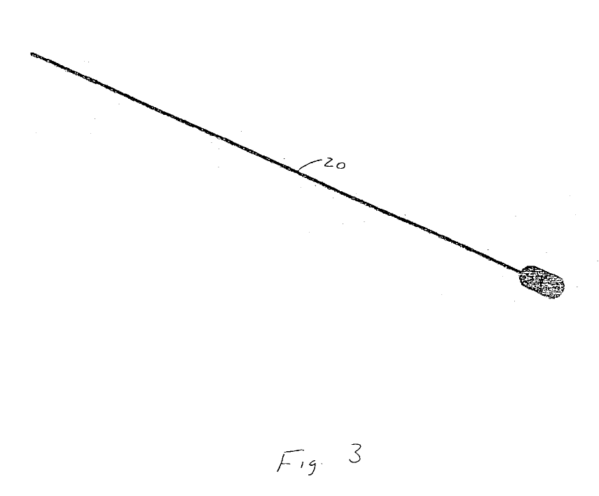

In use, the tip is affixed to the tube, and the tube is inserted through the nose, and through the esophagus and into the stomach. The stiffening stylet 20 shown in FIG. 3 may be inserted into the tube to push the tube, and direct the tube. The stiffening stylet cannot extend beyond the end of the tube, since the divider in the tip prevents travel of the stylet beyond the end of the tube. Further, a scope may be inserted with the tube, either traveling parallel to the tube, or inserted through the tube. The scope may be a fluoroscope or an endoscope. The scope gives visual information for guiding the tube by means of the stylet.

At such time as the tube enters the stomach, it must be directed to through the restriction created by the pylorus. Forceps or similar devices may be used to direct the tube. The tip receives the forceps, with each jaw of the forceps positioned in one of the notches at the tip. In this way, the tip is firmly held for directing the tube into the pylorus. Once the forward end of the tube has entered the pylorus, the stiffening stylet may be used to further direct the tube through the duodenum, and into the jejunum.

After the forward end of the tube is positioned in the jejunum, a liquid nutritional product may be directed into the tube. By directing food only into the smaller, longer tube, food reaches the jejunum, but is not directed into the stomach. Patients who are risk of aspirating food do not receive the liquid nutritional product into the stomach. Alternatively, the liquid food product may be directed into the longer, smaller diameter tube, and into the shorter, larger diameter tube, so that food reaches both the stomach and the jejunum. The shorter tube may be also used to decompress the stomach.

What is claimed is:

1. A feeding tube for nasal insertion, said feeding tube comprising an orifice near a forward end thereof, that is smaller than an inside diameter of said feeding tube, and wherein said forward end of said feeding tube has at least one opening therein formed on an outside wall of said tube that is in addition to said orifice.

2. A feeding tube for nasal insertion as described in claim 1, wherein said feeding tube has at least two openings therein formed on an outside wall of said tube that is in addition to said orifice.

3. A feeding tube for nasal insertion as described in claim 2, wherein said feeding tube has an additional opening at an end thereof.

4. A feeding tube for nasal insertion, said feeding tube comprising an orifice near a forward end thereof that is smaller than an inside diameter of said feeding tube, and wherein said forward end of said feeding tube has at least one opening therein formed on an outside wall of said tube that is in addition to said orifice and said at least one opening is downstream from said orifice.

5. A feeding tube for nasal insertion as described in claim 4, wherein said feeding tube has at least two openings therein formed on an outside wall of said tube that is in addition to said orifice and said at least two openings are downstream from said orifice.

6. A feeding tube for nasal insertion as described in claim 5, wherein said feeding tube has an additional opening at an end thereof.

7. A feeding tube for nasal insertion comprising an orifice near a forward end thereof that is smaller than an inside diameter of said tube, wherein said forward end of said tube extends beyond said orifice, and wherein said forward end that extends beyond said orifice has opposed openings therein.

8. A feeding tube for nasal insertion as described in claim 1, said feeding tube further comprising a stiffening stylet that slidably engages an interior of said feeding tube, and wherein said stiffening stylet is of sufficiently large size that it will not pass through said orifice.

9. A feeding tube for nasal insertion, said feeding tube comprising an orifice near a forward end thereof, and comprising a restriction that is present near said orifice and wherein said forward end of said feeding tube has at least one opening therein formed on an outside wall of said tube that is in addition to said orifice.

10. A feeding tube for nasal insertion as described in claim 9, wherein said feeding tube has at least two openings therein formed on an outside wall of said tube that is in addition to said orifice.

11. A feeding tube for nasal insertion as described in claim 10, wherein said feeding tube has an additional opening at an end thereof.

12. A feeding tube for nasal insertion as described in claim 9, said feeding tube further comprising a stiffening stylet that slidably engages an interior of said feeding tube, and wherein said stiffening stylet is of sufficiently large size that it will not pass through said restriction.
